(19) United States
(12) Patent Application Publication
(10) Pub. No.: US 2010/0112724 A1
(43) Pub. Date: May 6, 2010

(54) METHOD OF DETERMINATION OF PROTEIN LIGAND BINDING AND OF THE MOST PROBABLE LIGAND POSE IN PROTEIN BINDING SITE

(76) Inventors: Dmitry Gennadievich Tovbin, Moskovskaya oblast (RU); Dmitry Nikolaevich Tarasov, Moskovskaya oblast (RU)

Correspondence Address: LADAS & PARRY LLP, 26 WEST 61ST STREET, NEW YORK, NY, 10023, US

(21) Appl. No.: 12/594,845

(22) PCT Filed: Apr. 12, 2007
(86) PCT No.: PCT/RU07/00168

§ 371 Date: Oct. 6, 2009

(51) Int. Cl. G01N 33/566

(52) U.S. Cl. 436/501

(57) ABSTRACT

The present invention proposes a method of structural design, search and selection of potential medicinal compounds—ligands, comprising prognostication of the value of the protein ligand binding in terms of the score calculated with the help of the scoring function developed for scoring, and prognostication of the most probable ligand pose in the protein binding site in terms of the score calculated with the help of the scoring function developed for docking (the docking function). It is proposed to use two absolutely different scoring functions for docking and scoring. A special procedure is proposed for the development of docking function. Use of two absolutely different functions in the process of docking and scoring principally distinguishes the proposed method of predicting the binding affinity of ligand-protein interaction from all the known methods and makes it possible to substantially improve the quality of said prediction.

### FIELD OF THE ART

The present invention relates to medical chemistry and can be used in searching for medicinal compounds having required biological activity or function.

### STATE OF THE ART

There exists a large group of drugs which are relatively small chemical compounds capable of binding with definite proteins. It is known that the quality of this binding is defined by the free energy of the compound-protein interaction. The smaller the free energy, the stronger the interaction is.

One of the most important tasks in searching for new biologically active substances is to predict for the known protein and ligand the free energy of the ligand-protein interaction from the structure of the protein and the ligand. The free energy of the ligand-protein interaction can be calculated with absolute accuracy only if we know the energy of the ligand-protein interaction in all possible poses of the ligand relative to the protein, with taking into account the internal energy of the ligand and of the protein, and also the energy of the ligand and protein interaction with the environment [1]. However, only the pose of the ligand, in which the energy of the ligand-protein interaction is minimum and poses in immediate proximity therefrom define the free energy of the ligand-protein interaction. In such native pose the ligand is found with maximum probability. Experimentally, the ligand is detected in immediate proximity from the native pose. For these reasons in the majority of the known scoring methods the value of the score SS is calculated univocally for the native pose of the ligand relative to the protein binding site. In correct scoring the value of the score calculated in the native pose must be proportional to the free energy of the ligand-protein interaction:

SS˜ΔG (1)

For scoring according to such procedure it is necessary to know the native pose of the ligand. Sometimes this pose is known experimentally, for instance, from X-ray structures. Docking comprises methods of predicting the most probable pose of the ligand in the protein binding site. Usually, in the process of docking in different poses of the ligand relative to the binding site, the values of a certain score SD are calculated and such ligand pose is selected, in which the value of the score is minimum. In correct docking the minimum value of the score must be in the native pose:

SDₙ<SD (2)

where
SDₙ is the value of the score in the native pose of the ligand in the binding site,
SD is the value of the score in any other poses of the ligand in the binding site.

Scoring function with the help of which the score is calculated for scoring and docking function—scoring function with the help of which the score for docking is calculated in the general case are different. However in the majority of the known methods of predicting the native pose of the ligand in the protein binding site and the free energy of the ligand-protein interaction an approach has gained currency, when the same scoring functions which were usually developed for the scoring or for docking (GOLD [2], FLEX [3], GLIDE [4], ICM [5]) are used for docking and for scoring simultaneously.

This fact, in the authors' judgment, is one of the main sources of errors both in the prediction of the native pose of the ligand in the binding site and in the prediction of the free energy of the protein-ligand interaction.

### ESSENCE OF THE INVENTION

It is an object of the proposed invention to provide a new method of determining the native pose of the ligand in the binding site and of determining the free energy of the ligand-protein interaction, which method comprises developing docking function for the determination of the native pose of the ligand in the binding site and scoring function for the determination of the free energy of the ligand-protein interaction.

A distinctive feature of the proposed method is the use of two absolutely different functions for scoring and docking, namely, of scoring function specially developed for docking—docking function and scoring function specially developed for scoring.

It is proposed to develop docking function SD with the help of an initial docking function S₀ by the method of iterative docking of a certain set of ligands into a protein definite for each ligand, with the known native pose of the given ligand and modification of the docking function so that for each ligand from the given set among the set of the ligand poses, obtained as a result of docking at the preceding iteration, upon modification of the docking function the best score should be had by one of those poses which is disposed in immediate proximity from the native pose.

It is proposed to develop scoring function S₀ by modification so that the coefficient of correlation R² between the value of the score calculated in the native pose of the ligand in the protein binding site for a certain set of proteins with ligands and the experimentally known free energy of a given ligand with a given protein should be maximum.

The approach proposed in the present invention to the determination of the most probable ligand pose in the binding site and to the determination of the free energy of the ligand-protein interaction will become conditionally divided into: 1) docking the ligand into the active site of the protein with the help of docking function; 2) calculating the ligand score with the help of scoring function developed specially for scoring in the ligand pose with the best score obtained as a result of docking.

### The Terms and Definitions Used in the Present Description:

Ligand—molecule of an organic chemical compound with a molecular weight less than 500 a.u.
Ligand-protein binding—interaction of a ligand molecule with a protein molecule, leading to the formation of a stable molecular complex consisting of said molecules.
Protein binding site—definite place in a protein molecule, wherein ligand molecules become bound to the protein molecule.
Free energy of interaction ΔG—quantity determining the force of ligand-protein binding.
Active ligand—ligand interacting with a definite protein with a free energy less than −9 kcal/mol.
Native pose of ligand in protein binding site—the most probable pose of the ligand in the protein binding site.
Scoring—methods of predicting the free energy of the ligand-protein interaction.
Docking—methods of predicting the native pose of the ligand in the protein binding site.
Score S—the number defined by the structure of the ligand, by the structure of the binding site and depending on the pose of the ligand in the binding site.
Scoring functions—set of methods, functions and parameters with the help of which score S is calculated.
Scoring function for scoring SS—scoring function used for defining the free energy of the ligand-protein interaction. The value of the score calculated in the native pose must be proportional to the free energy of the ligand-protein binding.
Docking function SD—scoring function used for docking—for defining the native pose of the ligand in the protein binding site. The minimum value of the score in docking must correspond to the native pose of the ligand in the binding site.
Coefficient of correlation R²—the number characterizing the correlation between two sets of numbers. In the case of absence of correlation R²=0, in the case of total correlation R²=1.
Successful docking—docking, as a result of which the root-mean-square deviation between heavy atoms (RMSD) for the best-score pose of the ligand obtained as a result of docking and the native pose is less than 2 angströms.
Quality of docking D—ratio of successful dockings to the total number of dockings.

Therefore, the first aspect of the present invention relates to a method of selecting potential medicinal compounds—active ligands, comprising the determination of the native pose of the ligand in the binding site of the protein and the determination of the free energy of the ligand-protein interaction, which comprises the development of docking function for the determination of the native pose of the ligand in the binding site and of scoring function for the determination of the free energy of the ligand-protein interaction. Said method comprises the following steps:

a) selection of a set of experimental data about the pose of active ligands in the binding site of different proteins;

b) development of docking function SD by a method of iterative docking with simultaneous modification of the initial docking function S₀ in such a manner that for each protein structure from the set obtained in step a) among the set of the ligand poses obtained as a result of docking in the preceding interaction the minimum score should be had by one of those poses which is disposed in immediate proximity from the native pose of the ligand in the binding site;

c) development of scoring function for scoring SS by a method of modification of the initial scoring function S₀ in such a manner that the coefficient of correlation R² between the value of the score calculated in the native pose of the ligand in the protein binding site for the set of the proteins with the ligands, obtained in step a) and the known free energy of the interaction of a given ligand with a given protein should be maximum;

d) determination of the native pose of the ligand in the protein binding site by docking the ligand into the active site of the protein with the help of the docking function SD developed for docking in step b);

e) calculation of the score of the ligand with the help of the scoring function SS developed for scoring in step c), in the pose of the ligand, obtained as a result of docking n step d);

f) carrying out virtual screening of the ligands by repeating steps d) and e);

g) selection of active ligands among the ligands, i.e., ligands with the minimum value of the score and carrying out measurement of the free energy of binding until active ligands with the free energy of binding less than −9 kcal/mole are revealed.

According to the preferred embodiment, the inventive method contemplates that the score has the following form:

$$S = {\sum }_{i , j} {S}_{A , B} ( {r}_{i , j} ) + {S}_{0}$$

where
i and j are the numbers of atoms in the protein and in the ligand,
A and B denote the types of the protein and of the ligand,
r_{i,j} is the distance between them,
S₀ is a certain constant.

Besides, the method of the invention contemplates that the score between atoms of different types is approximated by the following function

$$S ( r ) = \{ \begin{matrix}e + {k ( r - {r}_{1} )}^{4} , & r < {r}_{1} \\ \frac{2 e}{{( {r}_{2} - {r}_{1} )}^{3}} {( r - {r}_{2} )}^{2} ( r - 1.5 {r}_{1} + 0.5 {r}_{2} ) , & {r}_{1} < r < {r}_{2} \\ 0 , & r > {r}_{2}\end{matrix}$$

wherein
the score is continuous and differentiable for any r>0;
the parameters e, r₁, r₂, k for each pair of types A and B vary in the course of score modification.

According to the preferred embodiment, the method of the invention contemplates that the following typification is used in it for the atoms of proteins and ligands:
carbons in SP₃ hybridization;
carbons in SP₂ hybridization;
halogens (F, Cl, Br, I);
atoms which can behave simultaneously as hydrogen donors and acceptors in the hydrogen bond (oxygen in OH group);
hydrogen acceptors in the hydrogen bond (for instance, oxygen in C═O or CO₂ group);
hydrogen donors in the hydrogen bond (for instance, nitrogen in NH₃ group);
metals in the protein binding site;
the interaction of hydrogens in explicit form is not considered.

In accordance with the method of the invention, the development of the docking function SD comprises the following steps:

i) carrying out docking for each ligand-protein complex with the initial docking function S₀ and selecting several hundreds of ligand poses with the minimum score, obtained as a result of docking;

ii) modification of the initial docking function S₀ in such s manner that for each structure of the protein with the native pose of the ligand among hundreds of the ligand poses obtained as a result of docking carried out in step i) the minimum score should be had by one of those poses which is disposed in the immediate proximity from the native pose;

iii) repeating steps i) and ii) several times with the docking function obtained in the preceding iteration.

Further in the inventive method in the course of virtual screening docking for ligands is effected with the help of a program of fractal search for the optimum score ligand pose, which program operates according to the following algorithm:

1) in the binding site several thousands of points, i.e., of such places where in principle any of the ligand atoms can be found are defined.

2) the ligand is posed in the protein binding site in a random manner, so that one of the ligand atoms should be found in an active point, local minimization of the ligand in terms of the score is carried out, and the score in the minimized pose is calculated. (this procedure is repeated several thousand times);

3) the minimized ligand poses disposed in immediate proximity from one another are combined into clusters, and one pose with the best score is selected from each cluster;

4) for each ligand pose obtained in the preceding step, the internal state is varied at random with a certain parameter a, and the pose in the binding site is varied at random with a certain parameter b; for each new pose local minimization is carried out and the score in the minimized pose is calculated; (this procedure is repeated several tens of times);

5) steps 2) and 3) are repeated several times, the parameters a and b are varied according to power laws aₙ₊₁=aₙ^{0.5}, b_{n+!}=bₙ^{0.5}.

The power-law iterative variation of parameters is typical of algorithms with the participation of fractal structures. To accelerate calculations, the score was calculated with the help of a grid—precalculated potentials for each type of the ligand atom in the protein binding site. Docking was effected into the protein whose structure did not vary in the course of docking and was the same as in the original complex from the PDB. Before docking, three-dimensional ligand structures were generated with the help of special program CORINA [10].

The program of docking is tested in the following manner: the known three-dimensional structures of the ligand in the protein binding site are taken, this ligand is removed, docking into the binding site of the removed ligand is effected, and the initial (native) pose of the ligand is compared with the pose obtained as a result of docking; in all tests of the program the non-coincidence of the native ligand pose with the ligand pose obtained as a result of docking is construed to be conditioned only by that the latter pose has a better score than any pose in the immediate proximity from the native pose, that is, the algorithm of searching for the best ligand pose in the overwhelming majority of cases operated properly, and all failures in docking stemmed from the not quite correct score.

The method according to the invention contemplates that the scoring function SS are developed with the help of the algorithm of developing the scoring function for scoring, described in step c), using scoring function S₀ as the initial ones, on a training set of 82 complexes, for which purpose testing is performed by the method of 10-fold cross-validation.

Besides, according to the claimed method, the coefficients of correlation R² between the value of the score calculated in the native pose and the experimentally known free energy of the interaction, for the initial scoring function S₀ and for the scoring function S₁ developed specially for scoring, have values presented below in Table 2.

Further, the method of the invention contemplates that docking function SD are developed with the help of the algorithm of developing docking function, described in step b), using docking function S₀ as the initial ones, on a training set of 82 complexes, for which purpose testing is performed by the method of threefold cross-validation.

Further, the inventive method contemplates that docking of the ligand into the binding site is effected with the help of docking function SD developed for docking in step b); wherein for the ligand pose with the minimum score obtained as a result of docking with the value of the score of docking SD₁ with the help of the scoring function developed for scoring in strep c), the value of score SS₁ is calculated; and wherein for all poses obtained as a result of docking the value of the score is calculated from the formula

Sᵢ=SDᵢ−SD₁+SS₁ (3)

And, finally, in the method according to the invention the values of the scores depending on the free energy of binding for each complex for the minimum ligand pose with respect to the score, obtained as a result of docking with the scoring function SS obtained in step c) with the scoring function SD obtained in step b) and docking with the scoring function SD and recalculating the score with the scoring function SS using the formula (3), are presented below in FIG. 2 and in Table 2.

The invention will further be described in more detail, with examples of embodying the invention; said examples are presented exclusively by way of illustration and cannot be used for limiting the scope of the inventors' claims.

### BRIEF DESCRIPTION OF THE FIGURES AND TABLES

Table 1 lists pdb codes and free energy of ligand-protein binding values for test and training protein-ligand complexes.

In Table 2:
R² is the coefficient of correlation between the value of the score calculated in the native pose and the experimentally known binding affinity (free energy of interaction),
D—quality of docking,
R²_{best}—coefficients of correlation between the value of the score calculated in the best pose obtained as a result of docking and the experimentally known binding affinity (free energy of interaction),
S₀—docking and scoring were carried out with the initial scoring function S₀,
SS—docking and scoring were carried out with the initial scoring function SS developed specially for scoring,
SD—docking and scoring were carried out with the docking function SD,
SD+SS—docking was carried out with the docking function SD and scoring was carried out with the scoring function SS according to formula 3;
AutoDock—docking was carried out with the help of scoring function used in the AutoDock program,
FlexX—data have been calculated in accordance with the results of testing the FlexX program, taken from the web site of this program [3].

FIG. 1 illustrates the quality of docking D in the process of training (1) and of testing (2), the quality of training of scoring function D′ (3) and the coefficients of correlation R² between the value of the score calculated in the native pose and the experimentally known binding affinity (free energy of interaction) (4), depending on the iteration number in the process of developing the scoring function for docking.

FIG. 2 shows the values of scores depending on the experimental binding affinity for 82 complexes:
(a) for the best ligand pose with relation to score, obtained as a result of docking with scoring function SS,
(b) for the best ligand pose with relation to score, obtained as a result of docking with docking function SD,
(c) for docking with docking function SD and with recalculating the score with scoring function SS according to formula (3).

### DETAILED DESCRIPTION OF THE INVENTION

### Scoring Function

The results of numerical experiments have shown that with correct training, with the help of simple scoring function it is possible to obtain almost the same results as with more complicated functions; therefore the authors have realized a rather simple but rapid method of score calculation. Scoring function had the following general form:

$$S = {\sum }_{i , j} {S}_{A , B} ( {r}_{i , j} ) + {S}_{0}$$

where
i and j are the numbers of atoms in the protein and in the ligand,
A and B denote the types of the protein and of the ligand,
r_{i,j} the distance between the protein atoms and the ligand atoms,
S₀ is a certain constant.

The scoring function between atoms of different types had the following general form

$$S ( r ) = \{ \begin{matrix}e + k \frac{{10}^{5}}{{r}_{1}^{8}} {( r - {r}_{1} )}^{4} , & r < {r}_{1} \\ f ( r ) , & {r}_{2} > r > {r}_{1} \\ 0 , & r > {r}_{2}\end{matrix}$$

where f(r)=ar³+br³+cr+d u f(r₁)=e, f(r₂)=0, f′(r₁)=0, f′(r₂)=0.

The parameters e, r₁, r₂, k for each pair of types A and B varied in the course of score modification. The scoring functions are continuous and continuously differentiable for any r>0.

The following typification was used for the atoms of proteins and ligands:
aliphatic carbons
aromatic carbons
hydrogen donors and acceptors in the hydrogen bond simultaneously (oxygen in OH group)
hydrogen acceptors in the hydrogen bond (for instance, oxygen in C═O or CO₂ group)
hydrogen donors in the hydrogen bond (for instance, nitrogen in NH₃ group)
halogens (F, Cl, Br, I)
metals in the protein binding site.

The interaction of hydrogens in explicit form was not considered. As the results of numerical experiments have shown, consideration of hydrogens in explicit form though probably improves the score quality, albeit only slightly, while markedly complicates and slows down the calculations. Furthermore, in the structure of the binding site hydrogen is very often not present in the initial experimental data, while the problem of hydrogen positioning is not trivial and may be solved ambiguously by different programs.

### Training and Test Protein-Ligand Complexes

The authors used a set of 82 protein-ligand complexes with the known binding affinities (free energies of interaction) and the known native, most probable ligand pose in the binding site. The complexes were selected from the database PDB [6] among the structures described in publications [7, 8, 9], with taking into account the following criteria:
the ligand is small—the number of heavy atoms in the ligand is less than 50
the ligand is sufficiently rigid—the number of rotatable covalent bonds in the ligand is less than 10
the ligand interacts with the protein without the presence of other molecules, except water and zinc ions, in immediate proximity from the ligand
the ligand interacts with the protein without the participation of any metal ions, except zinc ions
there are no errors in the structure of the protein in immediate proximity from the ligand
   The selected complexes are listed in Table 1.

### Docking

Docking was carried out with the help of the algorithm of fractal search for the ligand pose with optimum score, developed by the authors:

1. In the binding site several thousands of points, i.e., of such places where in principle any of the ligand atoms can be found were defined.

2. The ligand was posed in the protein binding site in a random manner, so that one of the ligand atoms should be found in an active point, local minimization of the ligand in terms of the score was carried out, and the score in the minimized pose was calculated. (This procedure is repeated several thousand times);

3. The minimized ligand poses disposed in immediate proximity from one another were combined into clusters, and one pose with the best score was selected from each cluster;

4. For each ligand pose obtained in the preceding step, the internal state was varied at random with a certain parameter a, and the pose in the binding site was varied at random with a certain parameter b. The larger the parameters a) and b), the stronger the variations. For each new pose local minimization was carried out and the score in the minimized pose was calculated. Such procedure was repeated several tens of times.

5. Steps 2 and 3 were repeated several times, the parameters a and b were varied according to the power laws aₙ₊₁=aₙ^{0.5}, b_{n+!}=bₙ^{0.5}.

The power-law iterative variation of parameters is typical of algorithms with the participation of fractal structures. To accelerate calculations, the score was calculated with the help of a grid—precalculated potentials for each type of the ligand atom in the protein binding site. Docking was effected into the protein whose structure did not vary in the course of docking and was the same as in the original complex from the PDB. Before docking, three-dimensional ligand structures were generated with the help of special program CORINA [10].

### Scoring Function for Scoring SS

This function was developed by the authors, using the following procedure: the initial scoring function S₀ was modified so that the coefficient of correlation R² between the value of the score calculated by the modified scoring function in the native pose of the ligand in the protein binding site for the training protein-ligand set and the experimentally known free energy of the interaction of a given ligand with a given protein should be maximum.

### Docking Function SD

This function was developed by the authors, using the following procedure:

1. For each ligand-protein complex docking of the native ligand with the initial docking function S₀ was carried out and several hundreds of the best ligand poses obtained as a result of docking were selected.

2. The initial docking function `S₀ was modified so that for each structure of the protein with the native pose of the ligand among hundreds of the best poses of the ligand, obtained as a result of docking in the preceding step, the best score should be had by one of the poses located in immediate proximity from the native pose.

3. Steps 1 and 2 were repeated several times with the docking function obtained in the preceding iteration.

### Initial Set of Scoring and Docking Function S₀

The initial scoring and docking function employed in all numerical experiments was the simplest, but reasonable from the physical consideration, scoring and docking function S₀, which reflects only the hydrophilic and hydrophobic interatomic interactions: attraction with the energy of −0.1 kcal/mol between hydrophobic atoms; repulsion with the energy of 0.0 kcal/mol between hydrophobic and hydrophilic atoms; and hydrogen bond with the energy of −1 kcal/mol between hydrophilic atoms.

Docking with Scoring Function Developed for Scoring

Scoring function SS were developed with the help of the above-described algorithm of developing the scoring function for scoring, with the use of scoring function S₀ as the initial ones, on a training set of 82 complexes.

In the development of scoring function, testing was carried out by the method of tenfold cross-validation: all complexes were divided into 10 equal set; 1 set was used as the test set; the 9 remaining sets were used as the training sets, and this procedure was repeated 10 times, wherein each time the set of the complexes, not used earlier, served as the test set. The errors obtained in the process of each testing were combined.

Presented in Table 2 are the coefficients of correlation R² between the value of the score calculated in the native pose and the experimentally known free interaction energy for the initial scoring function S₀ and for the scoring function SS developed specially for scoring. As can be seen, for the scoring function S₀ proposed from reasonable physical considerations without any fitting coefficients, the coefficient of correlation R² is relatively low (0.21), as against the coefficient of correlation obtained in the development of the scoring function for scoring (0.72), but a noticeable correlation still exists. It can be seen also that the coefficient of correlation R² for the scoring function SS (0.72) is as high as in the majority of the known scoring functions in the case when validation was carried out on the test complexes proposed by the authors of the scoring function, for example R²=0.7 for FlexX [3].

With the help of the scoring function S₀ and SS docking was carried out for 82 protein-ligand complexes. The docking was regarded to be successful, if the root-mean-square deviation RMSD between heavy atoms for the best ligand pose in terms of the score, obtained as a result of docking and the native pose was less than 2 angströms. Shown in Table 2 is the quality of docking D for docking with the initial scoring function S₀ and with the scoring function obtained as a result of development of the scoring function for scoring SS.

The quality of docking D for the initial scoring function S₀, proposed from reasonable physical considerations without any fitting coefficients, proved to be very high: 0.5. For the scoring function SS developed for scoring, the quality of docking D=0.34 proved to be worse than for the initial scoring function S₀. There is no direct relationship between the quality of scoring function in the process of scoring and the quality of the scoring performed with the help of the same scoring function. Indeed, in scoring the score must be proportional to the free energy of the ligand-score interaction (the score must satisfy condition (1)), while in docking the score must be proportional to the probability of the ligand to be in the definite pose (the score must satisfy condition (2)), that is, in the general case the scoring function used for scoring and the docking function used for docking are different.

In the case of virtual screening, docking and scoring of a large number of ligands into a definite binding site, the ligands selected as potentially interesting are those in which the score has the best value, the score being calculated in the pose obtained as a result of docking, and the latter pose, in the case of error in docking, may be located far away from the native pose. Presented in Table 2 are the coefficients of correlation R²_{best} between the value of the score calculated in the best pose obtained as a result of docking and the experimentally known free binding energy, for docking and scoring with the scoring function S₀ and SS. As can be seen, for all scoring functions the coefficients of correlation R², obtained on the same complexes, but for the native ligand pose rather than for the ligand pose obtained as a result of docking. This deterioration of the correlation is caused by errors in the docking.

For comparing the results obtained by the authors with the known programs, docking of the 82 test complexes was carried out with the aid of the docking program developed by the authors, but with the scoring function used in the program AutoDock [11], and the results of testing the program FlexX, presented by the authors of this program on the web site [3] were taken. In the programs AutoDock and FlexX the scoring function are empirical, the parameters of the scoring function were selected so as to minimize errors in predicting the free energy, according to a procedure resembling the one used by the authors in developing scoring function for scoring. In the program FlexX one and the same score was used for docking and for predicting free binding energy. In the program FlexX the quality of docking D˜0.7, while in docking with the scoring function AutoDock on the set of complexes proposed by us D˜0.6, this being somewhat better than for docking with the scoring function S₀ and SS. But both for docking with the scoring function AutoDock and for the program FlexX the coefficients of correlation R² _{best} proved to be very low for AutoDock (R²_{best}=0.16) and for FlexX (R²_{best}=0.05).

### Development of Score for Docking

Docking function SD were developed with the help of the above-described algorithm of developing docking function, using the docking function S₀ as the initial ones, on the training set of 82 complexes. In the development of docking function testing was effected by the method of 3-fold cross-validation.

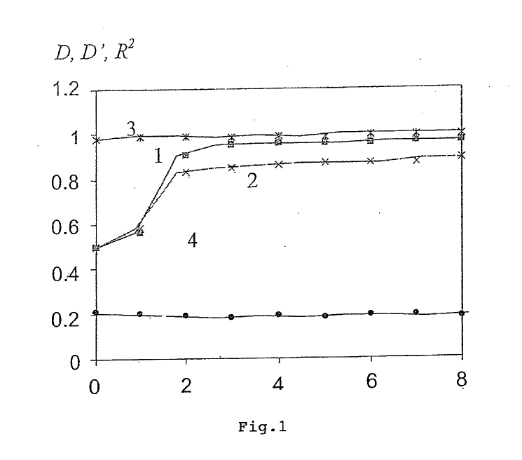

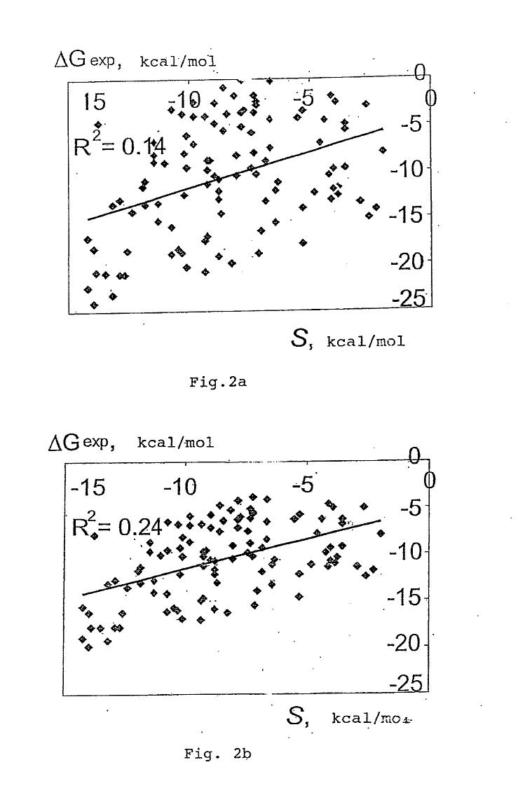

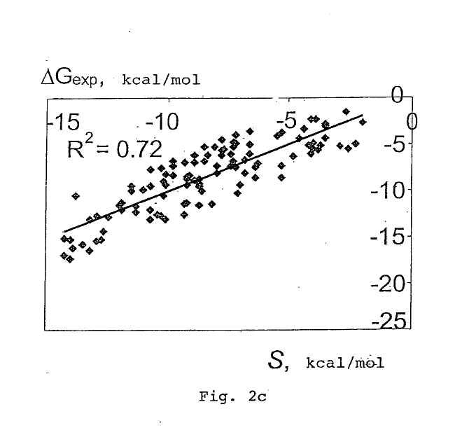

The quality of training the docking function D′ in a definite iteration in the course of their development is the ratio of the number of complexes for which after the modification of the docking function the best pose in terms of the score proved to be the pose with the RSMD for heavy atoms less than 2 angströms relative to the native pose to the total number of complexes. Shown in FIG. 1 are: the quality of docking D in the course of training (curve 1) and of testing (curve 2), the quality of training docking function D′ (curve 3), and the coefficients of correlation R² between the value of the score calculated in the native pose and the experimentally known free interaction energy (curve 4) depending on the number of the iteration in the course of development of the docking function. As can be seen, the quality of docking on the training complexes improves with each iteration and tends to the maximum possible value 1, almost reaching it (0.96). The quality of docking on the test complexes is slightly inferior than on the training complexes, reaching 0.89. The coefficients of correlation R² between the value of the score calculated in the native pose and the experimentally known free interaction energy do not improve and remains almost the same as for the initial scoring function S₀.

Thus, in the development of docking function with the help of the algorithm proposed by the authors it is possible to develop function, docking with which will be successful with a very high probability (96%), though errors in predicting free energy with these docking function may be unacceptably great.

Docking and Scoring with Different Scoring Functions

Successful prediction of the free ligand-protein interaction with the help of scoring function can be achieved only if the most probable pose of the ligand in the binding site is known. If the most probable pose of the ligand in the binding site is not known experimentally, it can be found with the help of docking. Docking function developed specially for docking but unsuitable for scoring and scoring function developed specially for scoring but unsuitable for docking can be combined in one method, and the most probable pose of the ligand in the binding site can be predicted with the help of the first ones, and in this pose the score can be calculated with the help of the second ones, that is, the free ligand-protein binding energy can thus be predicted. For these purposes the authors have proposed the following procedure:

1. Docking of the ligand into the binding site is performed with the help of docking function SD developed for docking.

2. For the best ligand pose in terms of the score obtained as a result of docking, with the value of the score of docking SD₁ with the help of scoring function developed specially for scoring, the value of the score SS₁ is calculated.

3. For all poses obtained as a result of docking the value of the score is calculated from the formula

Sᵢ=SDᵢ−SD₁+SS₁ (3)

The value of the score in the most probable pose obtained as a result of the docking calculated from the formula (3) will be equal to SS₁. If the best pose of the ligand found during docking coincides with the native pose, then in scoring with the score SS for the ligand in the native pose the value of the score would be SS₁. The values of the scores calculated from the formula (3) in all other poses i will be inferior to the value of the score SS, for the best pose found as a result of docking by ΔS=SDᵢ−SD₁, similarly to the case when the score would be calculated with the docking function SD and would be proportional to the probability of being found in the pose i.

For 82 complexes docking and scoring were carried out by following the above-described procedure: docking with score SD, scoring with score SS, and the final score was calculated from the formula (3). Presented in FIG. 2 are the values of the scores, depending on the experimental free binding energy for each complex for the best pose of the ligand in terms of the score, obtained as a result docking with the scoring function SS (FIG. 2a), with the docking function SD (FIG. 2b) and in docking with the docking function SD and in docking with the docking function SD and recalculating the score with the scoring function SS according to the formula (3) (FIG. 2c). These results are presented in Table 2. As can be seen, the best correlation of the score in the pose found as a result of docking with the experimental can be achieved only when docking is carried out with the docking function developed specially for docking and scoring is carried out according to the formula (3) with the scoring function specially developed for scoring.

The method in which docking was carried out employing scoring function of one type with subsequent recalculation of the score by employing other scoring function had also been used earlier, for instance, in the programs [2, 4]. The principle difference of the method of docking and scoring proposed by the authors from all other methods is that docking in the present method is carried out with the help of the docking function specially developed for docking, according to the procedure proposed by the authors and described above. Just owing to the high probability of successful docking (98%), the coefficient of correlation R²_{best} between the value of the score calculated in the best pose obtained as a result of docking and the experimentally known free binding energy in the present case is essentially greater than in other publications, though the coefficient of correlation R² between the value of the score calculated in the native pose and the experimentally known free interaction energy with the scoring function developed for scoring in the present case is almost the same as in other known scoring functions (0.7-0.8).

Hence:

It was shown that in the general case scoring function used for scoring and scoring function used for docking (docking function) are different scoring functions, and that scoring function successfully used in the process of scoring may yield unacceptable results in the process of docking and vice versa.

There was developed, described and tested a method of obtaining docking function intended exclusively for docking and it was shown that with the help of such docking function it is possible to achieve an almost 100% probability of predicting correct and most probable ligand pose in the active protein site.

It was shown that if docking is accomplished with the use of docking function, and for the best ligand pose in the active site, found as a result of such docking, scoring is accomplished with the use of scoring function specially developed for scoring, it is possible to predict the free ligand-protein interaction energy with considerably higher accuracy than in the case when rocking and scoring are accomplished with the help of scoring function not developed specially for these purposes.

### List of References

1. E M Lifshitz, L D Landau, Statistical Physics (Course of Theoretical Physics, Volume 5), Butterworth-Heinemann; 3th edition, 1984.
2. http://www.ccdc.cam.ac.uk/products/life sciences/gold/
3. http://www.biosolveit.de/FlexX/
4. http://www.schodinger.com/ProductDescription.php?mID=6&sID=6
5. http://www.molsoft.com/docking.html
6. http://www.pdb.org
7. Eldridge M., Murray C., Auton T., Paolini G., Mee R., “Empirical functions: I. the development of a fast empirical scoring function to estimate the affinity of ligands in receptor complexes, “J. Comp.-Aided Mol. Des. 11, 425-445, 1997.
8. Wang R., Lu Y., and Wang S., “Comparative Evaluation of 11 Scoring Functions for Molecular Docking”, J. Med. Chem., 46, 2287-2303, 2003.
9. Ishchenko A. V., Shakhnolvich E. I., “Small, molecule growth 2001 (SMoG2001): an improved knowledge-based scoring function for protein-ligand interactions”, J. Med. Chem. 45 (13): 2770-2780, 2002.
10. http://www.ol-net.de/index.html
11. http://autodock.scripps.edu/

TABLE 1

| | ΔG, kcal/ | | | | |
|---|---|---|---|---|---|
| Pdb | mol | Pdb code | ΔG, kcal/mol | Pdb code | ΔG, kcal/mol |
| 1A42 | −14 | 1DDS | −11.3 | 2DBL | −11.9 |
| 1ABE | −9.2 | 1DHF | −10.1 | 2DRI | −8.9 |
| 1ABF | −7.3 | 1DRF | −10.1 | 2GBP | −10.2 |
| 1ADD | −9.2 | 1DWB | −4 | 2QWB | −3.7 |
| 1AF2 | −4.2 | 1ETS | −11.5 | 2QWC | −4.8 |
| 1ANF | −7.3 | 1G6N | −6.8 | 2QWD | −6.6 |
| 1APB | −7.8 | 1HBP | −9.8 | 2QWE | −10.2 |
| 1BAP | −9.3 | 1HSL | −9.8 | 2QWF | −7.7 |
| 1BIT | −5.5 | 1JAO | −8.1 | 2QWG | −11.5 |
| 1BN1 | −13.2 | 1L83 | −4.6 | 2TMN | −8 |
| 1BN3 | −14 | 1MDQ | −7 | 2YPI | −6.6 |
| 1BN4 | −13.2 | 1MFE | −7.2 | 3FX2 | −12.7 |
| 1BNM | −14.2 | 1MNC | −12.6 | 3PTB | −6.3 |
| 1BNN | −14.2 | 1NNB | −7.2 | 3TMN | −8 |
| 1BNQ | −13.5 | 1NSC | −4.1 | 4HMG | −3.5 |
| 1BNT | −13.9 | 1NSD | −7.2 | 4TIM | −2.9 |
| 1BNU | −13.8 | 1PGP | −7.8 | 4TPI | −3.9 |
| 1BNV | −12.4 | 1PPC | −8.6 | 5ABP | −8.9 |
| 1BNW | −12.9 | 1PPH | −8.4 | 5ACN | −3.8 |
| 1BRP | −9.3 | 1RBP | −9.2 | 5TLN | −8.7 |
| 1BZM | −8.2 | 1RNT | −7.1 | 6ABP | −8.5 |
| 1CBX | −8.7 | 1TNG | −4 | 6GST | −6.4 |
| 1CIL | −12.9 | 1TNI | −2.3 | 6TIM | −4.4 |
| 1CIM | −12 | 1TNJ | −2.7 | 7ABP | −8.7 |
| 1CIN | −11.9 | 1TNK | −2 | 7TIM | −7.4 |
| 1CPS | −9.1 | 1TNL | −2.6 | 8ABP | −10.7 |
| 1DBJ | −10.4 | 2AK3 | −5.3 | 8ATC | −10.3 |
| 1DBK | −11 | 2CTC | −5.3 | 9ABP | −10.7 |

TABLE 2

| | R² | D | R²_{best} |
|---|---|---|---|
| S₀ | 0.21 | 0.50 | 0.14 |
| SS | 072 | 0.34 | 0.24 |
| SD | 0.14 | 0.98 | 0.14 |
| SD + SS | 0.72 | 0.98 | 0.72 |
| AutoDock | 0.7 | 0.64 | 0.16 |
| FlexX | 0.7 | 0.7 | 0.05 |

1. A method of selecting potential medicinal compounds—active ligands, comprising the determination the native pose of the ligand in the protein binding site and the determination of the protein ligand binding affinity, which comprises development of docking function for the determination of the native pose of the ligand in the binding site and development of scoring function for the determination of the protein ligand binding affinity, said method comprising the following steps:
a) selection of a set of experimental data about the pose of active ligands in the binding site of different proteins;
b) development of docking function SD by a method of iterative docking with simultaneous modification of the initial scoring function S₀ in such a manner that for each protein structure from the set obtained in step a) among the set of the ligand poses obtained as a result of docking in the preceding interaction the minimum score should be had by one of those poses which is disposed in immediate proximity from the native pose of the ligand in the binding site;
c) development of scoring function for scoring SS by a method of modification of the scoring function S₀ in such a manner that the coefficient of correlation R² between the value of the score calculated in the native pose of the ligand in the protein binding site for the set of the proteins with the ligands, obtained in step a) and the known free energy of the interaction of a given ligand with a given protein should be maximum;
d) determination of the native pose of the ligand in the protein binding site by docking the ligand into the active site of the protein with the help of the scoring function SD developed for docking in step b);
e) calculation of the score of the ligand with the help of the scoring function SS developed for scoring in step c), in the pose of the ligand, obtained as a result of docking n step d);
f) carrying out virtual screening of the ligands by repeating steps d) and e);
g) selection of active ligands among the ligands, i.e., ligands with the minimum value of the score and carrying out measurement of the free energy of binding until active ligands with the free energy of binding less than −9 kcal/mole are revealed.

2. The method of claim 1, in which the score has the following general form:
$$S = {\sum }_{i , j} {S}_{A , B} ( {r}_{i , j} ) + {S}_{0}$$
where
i and j are the numbers of atoms in the protein and in the ligand,
A and B denote the types of the protein and of the ligand,
r_{i,j} is the distance between them,
S₀ is a certain constant.

3. The method of claim 2, in which the score between the atoms of different types is approximated by the following function:
$$S ( r ) = \{ \begin{matrix}e + {k ( r - {r}_{1} )}^{4} , & r < {r}_{1} \\ \frac{2 e}{{( {r}_{2} - {r}_{1} )}^{3}} {( r - {r}_{2} )}^{2} ( r - 1.5 {r}_{1} + 0.5 {r}_{2} ) , & {r}_{1} < r < {r}_{2} \\ 0 , & r > {r}_{2}\end{matrix}$$
wherein
the score is continuous and differentiable for any r>0;
the parameters e, r₁, r₂, k for each pair of types A and B vary in the course of score modification.

4. The method of claim 2, in which the following typification is used in it for the atoms of proteins and ligands:
carbons in SP₃ hybridization;
carbons in SP₂ hybridization;
halogens (F, Cl, Br, I);
atoms which can behave simultaneously as hydrogen donors and acceptors in the hydrogen bond (oxygen in OH group);
hydrogen acceptors in the hydrogen bond (for instance, oxygen in C═O or CO₂ group);
hydrogen donors in the hydrogen bond (for instance, nitrogen in NH₃ group);
metals in the protein binding site;
the interaction of hydrogens in explicit form is not considered.

5. The method of claim 1, in which the development of the docking function SD comprises the following steps:
i) carrying out docking for each ligand-protein complex with the initial docking function S₀ and selecting several hundreds of ligand poses with the minimum score, obtained as a result of docking;
ii) modification of the initial docking function S₀ in such s manner that for each structure of the protein with the native pose of the ligand among hundreds of the ligand poses obtained as a result of docking carried out in step i) the minimum score should be had by one of those poses which is disposed in the immediate proximity from the native pose;
iii) repeating steps i) and ii) several times with the docking function obtained in the preceding iteration.

6. The method of claim 1, in which in the course of virtual screening docking for ligands is effected with the help of a program of fractal search for the optimum score ligand pose, which program operates according to the following algorithm:
1) in the binding site several thousands of points, i.e., of such places where in principle any of the ligand atoms can be found are defined.
2) the ligand is posed in the protein binding site in a random manner, so that one of the ligand atoms should be found in an active point, local minimization of the ligand in terms of the score is carried out, and the score in the minimized pose is calculated. (this procedure is repeated several thousand times);
3) the minimized ligand poses disposed in immediate proximity from one another are combined into clusters, and one pose with the best score is selected from each cluster;
4) for each ligand pose obtained in the preceding step, the internal state is varied at random with a certain parameter a, and the pose in the binding site is varied at random with a certain parameter b; for each new pose local minimization is carried out and the score in the minimized pose is calculated; (this procedure is repeated several tens of times);
5) steps 2) and 3) are repeated several times, the parameters a and b are varied according to power laws aₙ₊₁=aₙ^{0.5}, b_{n+!}=bₙ^{0.5}.

7. The method of claim 6, in which the program of docking is tested in the following manner: the known three-dimensional structures of the ligand in the protein binding site are taken, this ligand is removed, docking into the binding site of the removed ligand is effected, and the initial (native) pose of the ligand is compared with the pose obtained as a result of docking; in all tests of the program the non-coincidence of the native ligand pose with the ligand pose obtained as a result of docking is construed to be conditioned only by that the latter pose has a better score than any pose in the immediate proximity from the native pose.

8. The method of claim 1, in which the scoring function SS are developed with the help of the algorithm of developing the scoring function for scoring, described in step c), using scoring function So as the initial ones, on a training set of 82 complexes, for which purpose testing is performed by the method of 10-fold cross-validation.

9. The method of claim 1, in which the coefficients of correlation R² between the value of the score calculated in the native pose and the experimentally known free energy of the interaction, for the initial scoring function S₀ and for the scoring function S₁ developed specially for scoring, have values presented in Table 2.

10. The method of claim 1, in which docking function SD are developed with the help of the algorithm of developing docking function, described in step b), using docking function S₀ as the initial ones, on a training set of 82 complexes, for which purpose testing is performed by the method of 3-fold cross-validation.

11. The method of claim 1, in which docking of the ligand into the binding site is effected with the help of docking function SD developed for docking in step b); wherein for the ligand pose with the minimum score obtained as a result of docking with the value of the score of docking SD₁ with the help of the scoring function developed for scoring in strep c), the value of score SS₁ is calculated; and wherein for all poses obtained as a result of docking the value of the score is calculated from the formula
Sᵢ=SDᵢ−SD₁+SS₁ (3).

12. The method of claim 1, in which the values of the scores, depending on the free energy of binding for each complex for the minimum ligand pose with respect to the score, obtained as a result of docking with the scoring function SS obtained in step c) with the scoring function SD obtained in step b) and docking with the scoring function SD and recalculating the score with the scoring function SS using the formula (3), are presented in FIG. 2 and in Table 2.
